(19) United States
(12) Patent Application Publication
(10) Pub. No.: US 2019/0091128 A1
(43) Pub. Date: Mar. 28, 2019

(54) COMPOSITION OF GEL-GEL TYPE COMPRISING STABILIZED POLYMER PARTICLES

(71) Applicant: L'OREAL, Paris (FR)

(72) Inventors: Philippe ILEKTI, Chevilly Larue (FR); Laure DAUBERSIES, Chevilly Larue (FR); Christel LISON, Chevilly Larue (FR)

(21) Appl. No.: 15/537,422

(22) PCT Filed: Dec. 18, 2015
(86) PCT No.: PCT/EP2015/080607

§ 371 Date: Jun. 26, 2018

(30) Foreign Application Priority Data
Dec. 18, 2014 (FR) 1462725

(51) Int. Cl. A61K 8/81; A61Q 1/10; A61K 8/04; A61K 8/31

(52) U.S. Cl. CPC A61K 8/8152; A61K 8/31; A61K 8/042; A61Q 1/10

(57) ABSTRACT

The present invention relates to a composition, especially a cosmetic composition, comprising: at least one aqueous phase gelled with at least one hydrophilic gelling agent, said hydrophilic gelling agent being at least one nonionic associative polymer, and at least one oily phase gelled with at least one lipophilic gelling agent, said lipophilic gelling agent being at least one by drocarbon-based block copolymer, said oily phase also comprising at least one hydrocarbon-based oil and particles of at least one polymer surface-stabilized with a stabilizer; said phases forming therein a macro scopically homogeneous mixture; and said composition also comprising at least one hydrocarbon-based resin with a number-average molecular weight of less than or equal to 10 000 g/mol; and pentylene glycol. The present invention also relates to a process for preparing the same and to cosmetic processes for making up and/or caring for a keratin material, and also to the use of a dispersion of particles of at least one polymer surface-stabilized with a stabilizer in a non-aqueous medium containing at least one hydrocarbon-based oil, for preparing a mascara composition.

The present invention relates to the field of caring for and/or making up keratin materials, especially keratin fibres, and is more particularly directed towards proposing compositions, especially cosmetic compositions, which in particular give makeup or a care treatment gloss properties and persistence of the gloss after application, while at the same time limiting its migration (transfer resistance).

The term “keratin materials” preferably means human keratin materials, especially keratin fibres.

The present invention proves to be most particularly advantageous for caring for and/or making up keratin fibres.

The term “keratin fibres” in particular means the eyelashes and/or the eyebrows, and preferably the eyelashes. For the purposes of the present invention, this term. “keratin, fibres” also extends to synthetic false eyelashes.

In general, compositions intended for making up keratin fibres (mascara), for example the eyelashes, are of a nature to afford a matt makeup effect. The reason for this is that it is difficult to give them a capacity to afford a glossy film, given the lack of compatibility of the compounds conventionally considered for this purpose, in the field of making up the lips or the nails, and given the implementation imperatives required for making up the eyelashes.

Thus, the glossy appearance is conventionally afforded, in a cosmetic composition of lip gloss type, by the use of oily fatty substances, and in a composition of varnish type, by the use of rigid film-forming polymers.

However, the use of these two types of compound that are efficient for forming a glossy film impairs the drying properties as regards the oils and the comfort as regards the rigid film-foiiiiing polymers. Specifically, in the presence of oily fatty substances, the film deposited on the keratin materials does not dry, and the use of rigid film-foming polymers makes the deposit uncomfortable to users due to the perceived rigidity.

Moreover, users are nowadays more particularly interested in compositions, especially cosmetic compositions, that are free of surfactants.

The need thus remains for compositions, especially cosmetic compositions, especially mascaras, which have, after application, a very intense glossy makeup result, while at the same time affording good properties in terms of transfer resistance, comfort and persistence over time.

Contrary to all expectation, the inventors have found that the presence of at least one hydrocarbon-based oil as defined below, of at least specific particles of at least one stabilized polymer as defined below, of at least one specific hydrocarbon-based resin as defined below and of pentylene glycol, in a particular architecture in terms of the galenical formulation makes it possible precisely satisfy this need.

Thus, according to a first of its aspects, the present invention relates to a composition, especially a cosmetic composition, in particular for coating keratin materials, especially keratin fibres, more particularly the eyelashes, comprising:
at least one aqueous phase gelled with at least one hydrophilic gelling agent, said hydrophilic gelling agent being at least one nonionic associative polymer, preferably a nonionic polyurethane, and
at least one oily phase gelled with at least one lipophilic gelling agent, said lipophilic gelling agent being at least one hydrocarbon-based block copolymer; said oily phase also comprising at least one hydrocarbon-based oil and particles of at least one polymer that is surface-stabilized with a stabilizer, the polymer of the particles being a C1-C4 alkyl (meth)acrylate polymer; the stabilizer being an isobornyl (meth)acrylate polymer chosen from isobornyl (meth)acrylate homopolymer and statistical copolymers of isobomyl (meth)acrylate and of C1-C4 alkyl (meth)acrylate present in an isobomyl (meth)acrylate/C1-C4 alkyl (meth)acrylate weight ratio of greater than 4;
said phases forming therein a macroscopically homogeneous mixture; and said composition also comprising at least one hydrocarbon-based resin with a number-average molecular weight of less than or equal to 10 000 g/mol and pentylene glycol.

Thus, according to a preferred embodiment, the present invention relates to a composition, especially a cosmetic composition, in particular for coating keratin fibres, more particularly the eyelashes, comprising:
at least one aqueous phase gelled with at least one hydrophilic gelling agent, said hydrophilic gelling agent being at least one nonionic associative polymer, preferably a nonionic polyurethane; and
at least one oily phase gelled with at least one lipophilic gelling agent, said lipophilic gelling agent being at least one hydrocarbon-based block copolymer; said oily phase also comprising at least one hydrocarbon-based oil and particles of at least one polymer that is surface-stabilized with a stabilizer, the polymer of the particles being a C1 -C4 alkyl (meth)acrylate polymer; the stabilizer being an isobornyl (methjacrylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4; and at least one hydrocarbon-based resin with a number-average molecular weight of less than or equal to 10 000 g/mol;

said phases forming therein a macroscopically homogeneous mixture; and said composition also comprising pentylene glycol.

According to an advantageous variant, said particles are in dispersion in said non-aqueous medium containing at least one hydrocarbon-based oil.

Contrary to all expectation, the inventors have in fact found that the presence of at least one hydrocarbon-based oil as defined below, of at least specific particles of at least one stabilized polymer as defined below, of at least one specific hydrocarbon-based resin as defined below and of pentylene glycol in a galenical architecture that is in the faun of a macroscopically homogeneous mixture of a gelled aqueous phase and of a gelled oily phase as defined above, gives access to a mascara formulation which has expected properties in terms of persistence although containing a vehicle, and which can advantageously produce a glossy, intense, long-lasting, transfer-resistant and comfortable deposit.

The compositions according to the invention may especially be makeup compositions intended for affording the desired makeup effect, by their use alone on the eyelashes, but may also be non-pigmented or coloured compositions intended either to be superimposed on a makeup already deposited on the eyelashes or to be coated with a related makeup film: they are then termed, respectively, a top coat or a base coat. They may also be compositions intended for affording only care on keratin fibres and in particular the eyelashes.

Admittedly, “gel-gel” compositions have already been proposed in the cosmetics field. Formulations of this type combine a gelled aqueous phase with a gelled oily phase. Thus, gel/gel formulations are described in Almeida et al., Pharmaceutical Development and Technology, 2008, 13:487, tables 1 and 2, page 488; WO 99/65455; PI 0405758-9; WO 99/62497; JP 2005-112834 and WO 2008/081175.

Cosmetic compositions of gel-gel type comprising, as hydrophobic film-forming polymers, non-aqueous dispersions of polymers comprising polymer particles surface-stabilized with at least one stabilizer (these dispersions often being referred to as NADs (non-aqueous dispersions)) have also already been proposed. Such dispersions of surface-stabilized polymer particles may be manufactured as described in document WO 04/055081.

However, to the Inventors' knowledge, this type of composition does not comprise at least one hydrocarbon-based oil as defined below, at least specific particles of at least one stabilized polymer as defined below, at least one hydrocarbon-based resin as defined below and pentylene glycol.

According to another of its aspects, a subject of the invention is also a process for preparing a composition, especially a cosmetic composition in particular for coating keratin materials, preferably keratin fibres such as the eyelashes, comprising at least one step of mixing:
an aqueous phase gelled with at least one hydrophilic gelling agent, said hydrophilic gelling agent being at least one nonionic associative polymer, preferably a nonionic polyurethane; and
at least one oily phase gelled with at least one lipophilic gelling agent, said lipophilic gelling agent being at least one hydrocarbon-based block copolymer; said oily phase also comprising at least one hydrocarbon-based oil and particles of at least one polymer that is surface-stabilized with a stabilizer, the polymer of the particles being a C1 -C4 alkyl (meth)acrylate polymer; the stabilizer being an isobornyl (meth)acrylate polymer chosen from isobornyl (meth)acrylate homopolymer and statistical copolymers of isobomyl (meth)acrylate and of C1-C4 alkyl (meth)acrylate present in an isobomyl (meth)acrylate/C1-C4 alkyl (meth)acrylate weight ratio of greater than 4; under conditions suitable for obtaining a macroscopically homogeneous mixture;

said composition also comprising at least one hydrocarbon-based resin with a number-average molecular weight of less than or equal to 10 000 g/mol and pentylene glycol.

According to one embodiment variant, this process may advantageously comprise a step of mixing at least two or even more gelled phases.

For obvious reasons, the number of gelled aqueous phases and of gelled oily phases to be considered for forming a composition according to the invention may range for each of the two types of phase beyond two.

Advantageously, the mixing of the phases may be performed at room temperature (25° C.).

However, the process of the invention may comprise, if necessary, a step of heating the mixture.

According to one embodiment variant, the final formulation may be manufactured without following a particular order of introduction of the various constituents and, in certain cases, a “one-pot” manufacture may be performed.

According to a particular embodiment, the representative gelled phases of the same type of architecture are gelled with a different gelling agent.

Multi-phase formulations may thus be developed.

According to another of its aspects, a subject of the invention is also a process, especially a cosmetic process, for making up and/or caring for keratin materials, in particular keratin fibres, especially the eyelashes, comprising at least one step which consists in applying to said keratin materials a composition in accordance with the invention.

The present invention is also directed towards the use of a dispersion of particles of at least one polymer that is surface-stabilized with a stabilizer in a non-aqueous medium containing at least one hydrocarbon-based oil, the polymer of the particles being a C1-C4 alkyl (meth)acrylate polymer; the stabilizer being an isobornyl (meth)acrylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4 for preparing a mascara composition.

According to yet another of its aspects, the present invention relates to a process, especially a cosmetic process, for making up and/or caring for keratin materials, in particular keratin fibres, especially the eyelashes, comprising at least the application to said keratin materials of a macroscopically homogeneous composition obtained by extemporaneous mixing, before application or at the time of application to said keratin materials, of at least one aqueous phase gelled with at least one hydrophilic gelling agent, said hydrophilic gelling agent being at least one nonionic associative polymer, preferably a nonionic polyurethane, and at least one oily phase gelled with at least one lipophilic gelling agent, said lipophilic gelling agent being at least one hydrocarbon-based block copolymer, said oily phase also comprising at least one hydrocarbon-based oil and particles of at least one polymer surface-stabilized with a stabilizer, the polymer of the particles being a C1-C4 alkyl (meth)acrylate polymer; the stabilizer being an isobornyl (meth)acrylate polymer chosen from isobornyl (meth)acrylate homopolymer and statistical copolymers of isobornyl (meth)acrylate and of a C1-C4 alkyl (meth)acrylate present in an isobornyl (meth)acrylate/C1-C4 alkyl (meth)acrylate weight ratio of greater than 4; said composition also comprising at least one hydrocarbon-based resin with a number-average molecular weight of less than or equal to 10 000 g/mol; and pentylene glycol.

### Composition, In Particular Cosmetic Composition

To begin with, it is important to note that a composition according to the invention is different from an emulsion.

An emulsion generally consists of an oily liquid phase and an aqueous liquid phase. It is a dispersion of droplets of one of the two liquid phases in the other. The size of the droplets forming the dispersed phase of the emulsion is typically about a micrometre (0.1 to 100 μm). Furthermore, an emulsion requires the presence of a surfactant or of an emulsifier to ensure its stability over time.

In contrast, a composition according to the invention consists of a macroscopically homogeneous mixture of two immiscible gelled phases. These two phases both have a gel-type texture. This texture is especially reflected visually by a consistent and/or creamy appearance.

The term “macroscopically homogeneous mixture” means a mixture in which each of the gelled phases cannot be individualized by the naked eye.

More precisely, in a composition according to the invention, the gelled aqueous phase and the gelled oily phase interpenetrate and thus form a stable, consistent product. This consistency is achieved by mixing interpenetrated macrodomains. Thus, by microscope, the composition according to the invention is very different from an emulsion.

A composition according to the invention cannot be characterized either as having a “sense”, i.e. an O/W or W/O sense.

Thus, a composition according to the invention has a consistency of gel type. The stability of the composition is long-lasting without surfactant. Consequently, a composition, especially a cosmetic composition according to the invention does not require any surfactant or silicone emulsifier to ensure its stability over time.

Advantageously, a composition according to the invention is free of surfactants.

It is known practice from the prior art to observe the intrinsic nature of a mixture of aqueous and oily gels in a gel-type composition, for example, by introducing a dyestuff either into the aqueous gelled phase or into the lipophilic gelled phase, before the formation of the gel-type composition. During visual inspection, in a gel-type composition, the dyestuff appears uniformly dispersed, even if the dye is present solely in the gelled aqueous phase or in the gelled oily phase. Specifically, if two different dyes of. different colours are introduced, respectively, into the oily phase and into the aqueous phase, before formation of the gel-type composition, the two colours may be observed as being uniformly dispersed throughout the gel-type composition. This is different from an emulsion in which, if a dye, which is soluble in water or soluble in oil, is introduced, respectively, into the aqueous and oily phases, before forming the emulsion, the colour of the dye present will only be observed in the outer phase (Remington: The Science and Practice of Pharmacy, 19th Edition (1995), Chapter 21, page 282).

It is also known practice to distinguish a gel-type composition from an emulsion by perfouning a “drop test”. This test consists in demonstrating the bi-continuous nature of a gel-type composition. Specifically, as mentioned previously, the consistency of a composition is obtained by means of the interpenetration of the aqueous and oily gelled domains. Consequently, the bi-continuous nature of a gel-type composition may be demonstrated by means of a simple test with, respectively, hydrophilic and hydrophobic solvents. This test consists in depositing, firstly, one drop of a hydrophilic solvent on a first sample of the test composition, and, secondly, one drop of a hydrophobic solvent on a second sample of the same test composition, and in analysing the behaviour of the two drops of solvents. In the case of an O/W emulsion, the drop of hydrophilic solvent diffuses into the sample and the drop of hydrophobic solvent remains at the surface of the sample. In the case of a W/O emulsion, the drop of hydrophilic solvent remains at the surface of the sample and the drop of hydrophobic solvent diffuses throughout the sample. Finally, in the case of a gel-type composition (bi-continuous system), the hydrophilic and hydrophobic drops diffuse throughout the sample.

In the case of the present invention, the test that will be preferred for distinguishing a gel-type composition from an emulsion is a dilution test. Specifically, in a gel-type composition, the aqueous and oily gelled domains interpenetrate and form a consistent and stable composition, in which the behaviour in water and in oil is different from the behaviour of an emulsion. Consequently, the behaviour during dilution of a gel-type composition (hi-continuous system) may be compared to that of an emulsion.

More specifically, the dilution test consists in placing 40 g of product and 160 g of dilution solvent (water or oil) in a 500 mL plastic beaker. The dilution is performed with controlled stirring to avoid any emulsification. In particular, this is performed using a planetary mixer: Speed. Mixer TM DAC400FVZ. The speed of the mixer is set at 1,500 rpm for 4 minutes. Finally, observation of the resulting sample is performed using a light microscope at a magnification of ×100 (×10×10). It may be noted that oils such as Parleam® and Xiameter PMX-200 Silicone Fluid 5CS® sold by Dow Corning are suitable as dilution solvent, in the same respect as one of the oils contained in the composition.

In the case of a gel-type composition (hi-continuous system), when it is diluted in oil or in water, a heterogeneous appearance is always observed. When a gel-type composition (bi-continuous system) is diluted in water, pieces of oily gel in suspension are observed, and when a gel-type composition (bi-continuous system) is diluted in oil, pieces of aqueous gel in suspension are observed.

In contrast, during dilution, emulsions have a different behaviour. When an O/W emulsion is diluted in an aqueous solvent, it gradually reduces without having a heterogeneous and lumpy appearance. This same O/W emulsion, on dilution with oil, has a heterogeneous appearance (pieces of O/W emulsion suspended in the oil). When a W/O emulsion is diluted with an aqueous solvent, it has a heterogeneous appearance (pieces of W/O emulsion suspended in the water). This same W/O emulsion, when diluted in oil, gradually reduces without having a heterogeneous and lumpy appearance.

In a preferred embodiment, the composition comprises less than 5% surfactant, better still less than 2%, or even less than 1% and free of surfactant.

According to the present invention, the aqueous gelled phase and the oily gelled phase forming a composition according to the invention are present therein in a weight ratio ranging from 10/90 to 50/50. More preferentially, the aqueous phase and the oily phase are present in a weight ratio ranging from 20/80 to 40/60.

The ratio between the two gelled phases is adjusted according to the desired cosmetic properties.

A composition according to the invention has a viscosity preferentially ranging from 0.5 to 50 Pa.s, measured at a room temperature of 25° C. using a Rheomat RM 100® rheometer.

### Solids Content

The composition according to the invention advantageously comprises a solids content of greater than or equal to 25%, preferably 30%, better still 35%, in particular 40%, or even 42%, and preferentially 45%.

For the purposes of the present invention, the “solids content” denotes the content of non-volatile matter.

The solids content (abbreviated as SC) of a composition according to the invention is measured using a “Halogen Moisture Analyzer HR 73” commercial halogen desiccator from Mettler Toledo. The measurement is performed on the basis of the weight loss of a sample dried by halogen heating, and thus represents the percentage of residual matter once the water and the volatile matter have evaporated off.

This technique is fully described in the machine documentation supplied by Mettler Toledo.

The measuring protocol is as follows:

Approximately 2 g of the composition, referred to hereinbelow as the sample, are spread out on a metal crucible, which is placed in the halogen desiccator mentioned above. The sample is then subjected to a temperature of 105° C. until a constant weight is obtained. The wet mass of the sample, corresponding to its initial mass, and the dry mass of the sample, corresponding to its mass after halogen heating, are measured using a precision balance.

The experimental error associated with the measurement is of the order of plus or minus 2%.

The solids content is calculated in the following manner:

Solids content (expressed as weight percentage)=100×(dry mass/wet mass).

### Hydrophilic Gelling Agent

For the purposes of the present invention, the term “hydrophilic gelling agent” means a compound that is capable of gelling the aqueous phase of the compositions according to the invention.

The hydrophilic gelling agent is thus present in the aqueous phase of the composition.

The gelling agent may be water-soluble or water-dispersible.

As stated above, the aqueous phase of a composition according to the invention is gelled with at least one hydrophilic gelling agent, said hydrophilic gelling agent being at least one nonionic associative polymer.

Nonionic associative polymers are included among synthetic polymeric gelling agents.

For the purposes of the invention, the tem), “synthetic” means that the polymer is neither naturally existing nor a derivative of a polymer of natural origin.

For the purposes of the present invention, the term “associative polymer” means any amphiphilic polymer comprising in its structure at least one fatty chain and at least one hydrophilic portion. The associative polymers in accordance with the present invention are nonionic.

### Nonionic Associative Polymers

The nonionic associative polymers may be chosen from associative polyurethanes.

Associative polyurethanes are nonionic block copolymers comprising in the chain both hydrophilic blocks usually of polyoxyethylene nature (polyurethanes may also be referred to as polyurethane polyethers), and hydrophobic blocks that may be aliphatic sequences alone and/or cycloaliphatic and/or aromatic sequences.

In particular, these polymers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be envisaged. In addition, the polymer may comprise a hydrocarbon-based chain at one end or at both ends of a hydrophilic block.

Associative polyurethanes may be block polymers, in triblock or multiblock form. The hydrophobic blocks may thus be at each end of the chain (for example: triblock copolymer containing a hydrophilic central block) or distributed both at the ends and in the chain (for example: multiblock copolymer). These polymers may also be graft polymers or star polymers. Preferably, the associative polyurethanes are triblock copolymers in which the hydrophilic block is a polyoxyethylene chain comprising from 50 to 1000 oxyethylene groups. In general, associative polyurethanes comprise a urethane bond between the hydrophilic blocks, whence arises the name.

According to one preferred embodiment, a nonionic associative polymer of polyurethane type is used as gelling agent.

As examples of nonionic fatty-chain polyurethane polyethers that may be used in the invention, it is also possible to use Rheolate® FX 1100 (Steareth-100/PEG 136/HDI (hexamethyl diisocyanate) copolymer), Rheolate® 205 containing a urea function, sold by the company Elementis, or Rheolate® 208, 204 or 212, and also Acrysol® RM 184 or Acrysol® RM 2020.

Mention may also be made of the product Elfacos® T210 containing a C₁₂-C₁₄ alkyl chain, and the product Elfacos® T212 containing a C₁₆₋₁₈ alkyl chain (PPG-14 Palmeth-60 Hexyl Dicarbamate), from Akzo.

The product DW 1206B® from Rohm & Haas containing a C₂₀ alkyl chain and a urethane bond, sold at a solids content of 20% in water, may also be used.

Use may also be made of solutions or dispersions of these polymers. Examples of such polymers that may be mentioned are Rheolate® 255, Rheolate® 278 and Rheolate® 244 sold by the company Elementis. The products DW 1206F and DW 1206J sold by the company Rohm & Haas may also be used.

The associative polyurethanes that may be used according to the invention are in particular those described in the article by G. Fonnum, J. Bakke and Fk. Hansen, Colloid Polym. Sci., 271, 380-389 (1993).

Even more particularly, according to the invention, use may also be made of an associative polyurethane that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold in particular by the company Rohm & Haas under the names Aculyn® 46 and Aculyn® 44. Aculyn® 46 is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%), and Aculyn® 44 is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%).

Use may also be made of solutions or dispersions of these polymers. Examples of such polymers that may be mentioned include SER AD FX1010, SER AD FX1035 and SER AD 1070 from the company Elementis. Use may also be made of the products Aculyn® 44, Aculyn® 46, DW 1206F and DW 1206J, and also Acrysol° RM 184 from the company Rohm & Haas, or alternatively Borchi Gel LW 44 from the company Borchers, and mixtures thereof.

According to a particularly preferred embodiment, use is made, as hydrophilic gelling agent, of a nonionic associative polymer of fatty-chain nonionic polyurethane polyether type sold especially under the name Rheolate® FX 1100 (Steareth-100/PEG 136/HDI(hexamethyl diisocyanate) copolymer) by the company Elernentis.

The nonionic associative polymer(s) are advantageously used in a proportion of from 0.5% to 15% by weight of solids, preferably between 1% and 10% by weight and even more preferentially between 1% and 6% by weight, relative to the total weight of the composition.

### Lipophilic Gelling Agent

For the purposes of the present invention, the term “lipophilic gelling agent” means a compound that is capable of gelling the oily phase of the compositions according to the invention.

The lipophilic gelling agent is thus present in the oily phase of the composition.

The gelling agent is liposoluble or lipodispersible.

As emerges from the foregoing, the gelled oily phase comprises at least one lipophilic gelling agent, said lipophilic gelling agent being at least one hydrocarbon-based block copolymer.

The hydrocarbon-based block copolymers are included among lipophilic polymeric gelling agents.

### Hydrocarbon-Based Block Copolymers

The hydrocarbon-based block copolymers of the invention, also known as block copolymers, are preferably soluble or dispersible in the oily phase.

The hydrocarbon-based block copolymer may especially be a diblock, triblock, multiblock, radial or star copolymer, or mixtures thereof.

Such hydrocarbon-based block copolymers are described in patent application US-A-2002/005562 and in patent U.S. Pat. No. 5,221,534.

The copolymer may contain at least one block whose glass transition temperature is preferably less than 20° C., preferably less than or equal to 0° C., preferably less than or equal to −20° C. and more preferably less than or equal to −40° C. The glass transition temperature of said block may be between −150° C. and 20° C. and especially between −100° C. and 0° C.

The hydrocarbon-based block copolymer present in the composition according to the invention may be an amorphous copolymer fanned by polymerization of an olefin. The olefin may especially be an elastomeric ethylenically unsaturated monomer.

The term “amorphous polymer” means a polymer that does not have a crystalline form.

Examples of olefins that may be mentioned include ethylenic carbide monomers, especially containing one or two ethylenic unsaturations and containing from 2 to 5 carbon atoms, such as ethylene, propylene, butadiene, isoprene or pentadiene.

Advantageously, the hydrocarbon-based block copolymer is an amorphous block copolymer of styrene and of an olefin.

Block copolymers comprising at least one styrene block and at least one block comprising units chosen from butadiene, ethylene, propylene, butylene and isoprene or a mixture thereof are especially preferred.

According to one preferred embodiment, the hydrocarbon-based block copolymer is hydrogenated to reduce the residual ethylenic unsaturations after the polymerization of the monomers.

In particular, the hydrocarbon-based block copolymer is an optionally hydrogenated copolymer, containing styrene blocks and ethylene/C₃-C₄ alkylene blocks.

According to one preferred embodiment, the composition according to the invention comprises at least one diblock copolymer, which is preferably hydrogenated, preferably chosen from styrene-ethylene/propylene copolymers, styrene-ethylene/butadiene copolymers and styrene-ethylene/butylene copolymers. Diblock polymers are especially sold under the name Kraton® G1701E by the company Kraton Polymers.

According to another preferred embodiment, the composition according to the invention comprises at least one triblock copolymer, which is preferably hydrogenated, preferably chosen from styrene-ethylene/propylene-styrene copolymers, styrene-ethylene/butadiene-styrene copolymers, styrene-isoprene-styrene copolymers and styrene-butadiene-styrene copolymers. Triblock polymers are especially sold under the names Kraton® G1650, Kraton® Di 101, Kraton® D1102 and Kraton® D1160 by the company Kraton Polymers.

According to one embodiment of the present invention, the hydrocarbon-based block copolymer is a styrene-ethylene/butylene-styrene triblock copolymer.

According to one preferred embodiment of the invention, it is especially possible to use a mixture of a styrene-butylene/ethylene-styrene triblock copolymer and of a styrene-ethylene/butylene diblock copolymer, especially the products sold under the name Kraton® 01657M by the company Kraton Polymers.

According to another preferred embodiment, the composition according to the invention comprises a mixture of styrene-butylene/ethylene-styrene hydrogenated triblock copolymer and of ethylene-propylene-styrene hydrogenated star polymer, such a mixture possibly being especially in isododecane or in another oil. Such mixtures are sold, for example, by the company Penreco under the trade names Versagel° M5960 and Versagel° M5670.

Advantageously, a diblock copolymer such as those described previously is used as hydrocarbon-based block copolymer, in particular a styrene-ethylene/propylene diblock copolymer or a mixture of diblock and triblock copolymers, as described previously.

The hydrocarbon-based block copolymer(s) may be present in a content ranging from 0.5% to 15% by weight, relative to the total weight of the composition, preferably ranging from 1% to 10% by weight and even more advantageously from 2% to 8% by weight, relative to the total weight of the composition.

### Hydrophilic Gelling Agent/Lipophilic Gelling Agent System

As stated previously, a composition according to the invention comprises as hydrophilic gelling agent(s) at least one nonionic associative polymer.

As preferred nonionic associative polymers, mention may be made more particularly of the nonionic associative polymers especially of polyurethane type, for instance associative polyurethanes, in particular fatty-chain nonionic polyurethane polyethers such as Steareth-100/PEG-136/HDI copolymer sold under the name Rheolate FX 1100 by Elementis.

As stated previously, a composition according to the invention comprises as lipophilic gelling agent(s) at least one hydrocarbon-based block copolymer.

As preferred hydrocarbon-based block copolymers, mention may be made of copolymers bearing styrene blocks and ethylene/C₃-C₄ alkylene blocks, which are preferably hydrogenated, such as:
diblock copolymers, which are preferably hydrogenated, chosen especially from styrene-ethylene/propylene copolymers, styrene-ethylene/butadiene copolymers, styrene-ethyleneibutylene copolymers, such as the diblock polymers sold under the name Kraton® G1701E by the company Kraton Polymers.
triblock copolymers, which are preferably hydrogenated, preferably chosen from styrene-ethylene/propylene-styrene copolymers, styrene-ethylene/butadiene-styrene copolymers, styrene-isoprene-styrene copolymers and styrene-butadiene-styrene copolymers such as those sold under the names Kraton® G1650, Kraton® D1101, Kraton® D1102 and Kraton® D1160 by the company Kraton. Polymers,
mixtures of a styrene-butylene/ethylene-styrene triblock copolymer and of a styrene-ethylene/butylene diblock copolymer, especially the products sold under the name Kraton® G1657M by the company Kraton Polymers, and
mixtures of hydrogenated styrene-butylene/ethylene-styrene triblock copolymer and of hydrogenated ethylene-propylene-styrene star polymer, such as those sold by the company Penreco under the trade names Versagel®

M5960 and Versagel® M5670.

### Hydrocarbon-Based Resin

As stated previously, the claimed compositions comprise at least one hydrocarbon-based resin, especially as detailed hereinbelow.

For the purposes of the present invention, the term “hydrocarbon-based resin” means a compound comprising carbon and hydrogen atoms, including a compound comprising carbon, hydrogen and oxygen atoms.

This type of compound is particularly advantageous since it makes it possible not only to significantly increase the persistence over time, but also to give the film gloss after application of a composition according to the invention to keratin materials and more particularly to keratin fibres such as the eyelashes and/or the eyebrows.

Specifically, as demonstrated in the experimental section below, the deposit of a composition according to the invention produced on keratin fibres and especially the eyelashes remains glossy after application once dry.

In particular, said hydrocarbon-based resin(s) are present totally or partially, and preferably solely, in the gelled oily phase.

Preferably, the hydrocarbon-based resin used in the composition according to the invention has a number-average molecular weight of less than or equal to 10 000 g/mol, especially ranging from 250 to 5000 g/mol, better still less than or equal to 2000 g/mol and especially ranging from 250 to 2000 g/mol.

The number-average molecular weights (Mn) are determined by gel permeation liquid chromatography (THF solvent, calibration curve established with linear polystyrene standards, refractometric detector).

Resins that may be suitable for use in the invention are described especially in the Handbook of Pressure Sensitive Adhesive, edited by Donatas Satas, 3rd edition, 1989, pages 609-619.

The resin of the composition according to the invention may be chosen from rosin, rosin derivatives and hydrocarbon-based resins other than rosin and derivatives thereof, and mixtures thereof.

The resin of the composition according to the invention is preferably solid at 25° C.

Rosin is a mixture predominantly comprising organic acids known as rosin acids (mainly acids of abietic type and of pimaric type).

Three types of rosin exist: rosin (“gum rosin”) obtained by incision on live trees, wood rosin, which is extracted from pine wood or stumps, and tall oil (“tall oil rosin”), which is obtained from a by-product originating from the production of paper.

The rosin derivatives may be derived in particular from the polymerization, hydrogenation and/or esterification (for example with polyhydric alcohols such as ethylene glycol, glycerol or pentaerythritol) of rosin acids. Examples that may be mentioned include the rosin esters sold under the reference Foral 85, Pentalyn H and Staybelite Ester 10 by the company Hercules; Foral 105 Synthetic Resin by the company Pinova; Sylvatac 95 and Zonester 85 by the company Arizona Chemical, or Unirez 3013 by the company Union Camp.

The hydrocarbon-based resins, other than rosin and derivatives thereof, are chosen from low molecular weight polymers that may be classified, according to the type of monomer they comprise, as:
indene hydrocarbon-based resins, preferably such as resins derived from the polymerization in major proportion of indene monomer and in minor proportion of a monomer chosen from styrene, methylindene and methyistyrene, and mixtures thereof. These resins may optionally be hydrogenated. These resins may have a molecular weight ranging from 290 to 1,150 g/mol.

Examples of indene resins that may be mentioned include those sold under the reference Escorez 7105 by the company Exxon Chem., Nevchem 100 and Nevex 100 by the company Neville Chem., Norsolene S105 by the company Sartomer, Picco 6100 by the company Hercules and Resinall by the company Resinall Corp., or the hydrogenated indene/methylstyrene/styrene copolymers sold under the name “Regalite” by the company Eastman Chemical, in particular Regalite R 1100, Regalite R 1090, Regalite R-7100, Regalite R1010 Hydrocarbon Resin and Regalite R1125 Hydrocarbon Resin;
aliphatic pentanediene resins such as those derived from the majority polymerization of the 1,3-pentanediene (trans- or cis-piperylene) monomer and of minor monomer(s) chosen from isoprene, butene, 2-methyl-2-butene, pentene and 1,4-pentanediene, and mixtures thereof. These resins may have a molecular weight ranging from 1,000 to 2,500 g/mol.

Such 1,3-pentanediene resins are sold, for example, under the references Piccotac 95 by the company Eastman Chemical, Escorez 1304 by the company Exxon Chemicals, Nevtac 100 by the company Neville Chem. or Wingtack 95 by the company Goodyear;
mixed resins of pentanediene and of indene, which are derived from the polymerization of a mixture of pentanediene and indene monomers such as those described above, for instance the resins sold under the reference Escorez 2101 by the company Exxon Chemicals, Nevpene 9500 by the company Neville Chem., Hercotac 1148 by the company Hercules, Norsolene A 100 by the company Sartomer, and Wingtack 86, Wingtack Extra and Wingtack Plus by the company Goodyear;
diene resins of cyclopentanediene dimers such as those derived from the polymerization of a first monomer chosen from indene and styrene, and of a second monomer chosen from cyclopentanediene dimers such as dicyclopentanediene, methyldicyclopentanediene and other pentanediene dimers, and mixtures thereof. These resins generally have a molecular weight ranging from 500 to 800 g/mol, for instance those sold under the reference Betaprene BR 100 by the company Arizona Chemical Co., Neville LX-685-125 and Neville LX-1000 by the company Neville Chem., Piccodiene 2215 by the company Hercules, Petro-Rez 200 by the company Lawter or Resinall 760 by the company Resinall Corp.;
diene resins of isoprene dimers such as terpenic resins derived from the polymerization of at least one monomer chosen from α-pinene, α-pinene and limonene, and mixtures thereof. These resins may have a molecular weight ranging from 300 to 2000 g/mol. Such resins are sold, for example, under the names Piccolyte A115 and S125 by Hercules or Zonarez 7100 or Zonatac 105 Lite by Arizona Chem.

Mention may also be made of certain modified resins such as hydrogenated resins, for instance those sold under the name Eastotac C6-C20 Polyolefin by the company Eastman Chemical Co., under the reference Escorez 5300 by the company Exxon Chemicals, or the resins Nevillac Hard or Nevroz sold by the company Neville Chem., the resins Piccofyn A-100, Piccotex 100 or Piccovar AP25 sold by the company Hercules or the resin SP-553 sold by the company Schenectady Chemical Co.

According to a preferred embodiment, the resin is chosen from indene hydrocarbon-based resins, aliphatic pentadiene resins, mixed resins of pentanediene and of indene, diene resins of cyclopentanediene dimers and diene resins of isoprene dimers, or mixtures thereof.

Preferably, the composition comprises at least one compound chosen from the resins as described previously, in particular from indene hydrocarbon-based resins and aliphatic pentadiene resins, and mixtures thereof. According to a preferred embodiment, the resin is chosen from indene hydrocarbon-based resins.

According to a preferred embodiment, the resin is chosen from indene/methylstyrene/hydrogenated styrene copolymers.

In particular, use may be made of indene/methylstyrene/hydrogenated styrene copolymers, such as those sold under the name Regalite by the company Eastman Chemical, such as Regalite R 1100 CG Hydrocarbon Resin, Regalite R 1100, Regalite R 1090, Regalite R-7100, Regalite R1010 Hydrocarbon Resin and Regalite R1125 Hydrocarbon Resin.

A composition according to the invention may comprise from 3% to 30% by weight, preferably from 5% to 30% by weight and even more preferentially from 5% to 25% by weight of resin(s) relative to the total weight of the composition.

### Pentylene Glycol

As mentioned above, a composition according to the invention also comprises pentylene glycol, sold especially under the name 616751 Hydrolite-5® by the company Symrise.

Pentylene glycol is particularly advantageous since it provides gloss and contributes towards the persistence of the gloss after application of a composition according to the invention to keratin materials and more particularly to keratin fibres.

A composition according to the invention may comprise from 1% to 20% by weight, preferably from 1.5% to 15% by weight and even more preferentially from 2% to 10% by weight of pentylene glycol, relative to the total weight of the composition.

### Aqueous Phase

Besides the abovementioned pentylene glycol, the aqueous phase of a composition according to the invention comprises water and optionally a water-soluble solvent, other than the pentylene glycol that is suitable for use in the invention.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention. mention may be made especially of lower monoalcohols containing from 1 to 5 carbon atoms, such as ethanol and isopropanol.

The aqueous phase (water and optionally the water-miscible solvent) may be present in the composition in a content ranging from 10% to 50% and better still from 15% to 40% by weight relative to the total weight of said composition.

In particular, a composition according to the invention advantageously comprises a water content at least equal to 10% by weight, preferably at least equal to 15% by weight and preferentially ranging from 15% to 45% by weight, relative to the total weight of the composition.

### Hydrocarbon-Based Oil

The composition according to the invention comprises a hydrocarbon-based oil.

This oil may be volatile (vapour pressure greater than or equal to 0.13 Pa measured at 25° C.) or non-volatile (vapour pressure less than 0.13 Pa measured at 25° C.).

Preferably, the hydrocarbon-based oil is volatile.

The hydrocarbon-based oil is an oil (non-aqueous compound) that is liquid at room temperature (25° C.).

The team “hydrocarbon-based oil” means an oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

The hydrocarbon-based oil may be chosen from:

hydrocarbon-based oils containing from 8 to 16 carbon atoms, and especially:
branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade name Isopar or Permethyl,
linear alkanes, for instance n-dodecane (C₁₂) and n-tetradecane (C₁₄) sold by Sasol under the respective references Parafol 12-97 and Parafol 14-97, and also mixtures thereof, the undecane-tridecane mixture, the mixtures of n-undecane (C₁₁) and of n-tridecane (C13) obtained in Examples 1 and 2 of patent application WO 2008/155 059 from the company Cognis, and mixtures thereof,
short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have chain lengths varying from C₄ to C₂₄, these chains possibly being linear or branched, and saturated or unsaturated; these oils are especially heptanoic or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion-flower oil and musk rose oil; shea butter; or else caprylic/capric acid triglycerides, for instance those sold by the company Stearineries Dubois or those sold under the names Miglyol 810®, 812® and 818® by the company Dynamit Nobel,
synthetic ethers containing from 10 to 40 carbon atoms;
linear or branched hydrocarbons of mineral or synthetic origin, such as petroleum jelly, polydecenes, hydrogenated polyisobutene such as Parleamt, squalane and liquid paraffins, and mixtures thereof,
synthetic esters such as oils of formula R₁COOR₂ in which R₁ represents a linear or branched fatty acid residue containing from 1 to 40 carbon atoms and R₂ represents an, in particular, branched hydrocarbon-based chain containing from 1 to 40 carbon atoms, on condition that R₁+R₂ for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalkyl heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol;
mixtures thereof.

More particularly, the content of hydrocarbon-based oil(s) ranges from 30% to 75% by weight and preferably from 40% to 60% by weight relative to the total weight of the composition.

This hydrocarbon-based oil may be provided totally or partly with the surface-stabilized polymer particles, in particular when these particles are introduced into the composition in the form of a pre-prepared dispersion of stabilized polymer particles. In this case, the hydrocarbon-based oil present in the composition represents at least the non-aqueous medium of the dispersion of polymer particles.

Advantageously, the hydrocarbon-based oil is apolar (thus formed solely from carbon and hydrogen atoms).

The hydrocarbon-based oil is preferably chosen from hydrocarbon-based oils containing from 8 to 16 carbon atoms and better still from 12 to 16 carbon atoms, in particular the apolar oils described previously.

Preferentially, the hydrocarbon-based oil is isododecane. More particularly, the isododecane content ranges from 20% to 60% by weight, preferably from 25% to 55% by weight and even more preferentially from 30% to 50% by weight, relative to the total weight of the composition.

Preferably, the hydrocarbon-based oil(s), in particular isododecane, constitute the only oil(s) of the composition, or are present in a predominant weight content relative to the additional oil(s) that may be present in the composition.

Thus, according to a particular embodiment, the hydrocarbon-based oil(s) are present in a composition according to the invention in a content ranging from 20% to 60% by weight, preferably from 25% to 55% by weight and even more preferentially from 30% to 50% by weight relative to the total weight of the composition, the hydrocarbon-based oil(s) preferably being apolar, more preferentially volatile, even more preferentially containing from 8 to 16 carbon atoms, or even better still isododecane.

In accordance with a particular embodiment of the invention, if the composition contains one or more non-volatile oils, their content advantageously does not exceed 10% by weight, preferably does not exceed 5% by weight relative to the total weight of the composition, and better still does not exceed 2% by weight relative to the total weight of the composition, or even is free of non-volatile oil(s).

### Polymer Particles

The composition according to the invention moreover comprises particles, which are generally spherical, of at least one surface-stabilized polymer.

Preferably, the particles are introduced into the composition in the foilii of a dispersion of particles, which are generally spherical, of at least one surface-stabilized polymer, in an oily medium, advantageously containing at least one hydrocarbon-based oil, as defined previously.

The polymer of the particles is a C1-C4 alkyl (meth)acrylate polymer.

The C1-C4 alkyl (meth)acrylate monomers may be chosen from methyl (meth)acrylate, ethyl (meth)acrylate, n-propyl (meth)acrylate, isopropyl (meth)acrylate, n-butyl (meth)acrylate and tert-butyl (meth)acrylate.

A C1 -C4 alkyl acrylate monomer is advantageously used. Preferentially, the polymer of the particles is a methyl acrylate and/or ethyl acrylate polymer.

The polymer of the particles may also comprise an ethylenically unsaturated acid monomer or the anhydride thereof, chosen especially from ethylenically unsaturated acid monomers comprising at least one carboxylic, phosphoric or sulfonic acid function, such as crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and salts thereof.

Preferably, the ethylenically unsaturated acid monomer is chosen from (meth)acrylic acid, maleic acid and maleic anhydride.

The salts may be chosen from salts of alkali metals, for example sodium or potassium; salts of alkaline-earth metals, for example calcium, magnesium or strontium; metal salts, for example zinc, aluminium, manganese or copper; ammonium salts of formula NH₄⁺; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles may thus comprise or consist essentially of 80% to 100% by weight of C1 -C4 alkyl (rneth)acrylate and of 0% to 20% by weight of ethylenically unsaturated acid monomer, relative to the total weight of the polymer.

According to a first embodiment of the invention, the polymer consists essentially of a polymer of one or more C1-C4 alkyl (meth)acrylate monomers.

According to a second embodiment of the invention, the polymer consists essentially of a copolymer of C1-C4 (meth)acrylate and of (meth)acrylic acid or maleic anhydride.

The polymer of the particles may be chosen from:
methyl acrylate homopolymers
ethyl acrylate homopolymers
methyl acrylate/ethyl acrylate copolymers
methyl acrylate/ethyl acrylate/acrylic acid copolymers
methyl acrylate/ethyl acrylate/maleic anhydride copolymers
methyl acrylate/acrylic acid copolymers
ethyl acrylate/acrylic acid copolymers
methyl acrylate/maleic anhydride copolymers
ethyl acrylate/maleic anhydride copolymers.

Advantageously, the polymer of the particles is a non-crosslinked polymer.

The polymer of the particles preferably has a number-average molecular weight ranging from 2000 to 10 000 000 and preferably ranging from 150 000 to 500 000.

In the case of a particle dispersion, the polymer of the particles may be present in the dispersion in a content ranging from 21% to 58.5% by weight and preferably ranging from 36% to 42% by weight, relative to the total weight of the dispersion.

The stabilizer is an isobornyl (meth)acrylate polymer chosen from isobomyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4, preferably greater than 4.5 and even more advantageously greater than or equal to 5. Advantageously, said weight ratio ranges from 4.5 to 19, preferably from 5 to 19 and more particularly from 5 to 12.

Thus, according to a particular embodiment, a composition according to the invention comprises one or more stabilizers, said stabilizer(s) being a statistical copolymer of isobornyl (meth)acrylate and of C1-C4 alkyl (meth)acrylate present in an isobornyl (meth)acrylate/C1-C4 alkyl (meth)acrylate weight ratio of greater than or equal to 5.

Advantageously, the stabilizer is chosen from:
isobornyl acrylate homopolymers
statistical copolymers of isobornyl acrylate/methyl acrylate
statistical copolymers of isobornyl acrylate/methyl acrylate/ethyl acrylate
statistical copolymers of isobornyl methacrylate/methyl acrylate
in the weight ratio described previously.

The stabilizing polymer preferably has a number-average molecular weight ranging from 10 000 to 400 000 and preferably ranging from 20 000 to 200 000.

The stabilizer is in contact with the surface of the polymer particles and thus makes it possible to stabilize these particles at the surface, in particular in order to keep these particles in dispersion in the non-aqueous medium of the dispersion.

Advantageously, the combination of the stabilizer(s) +polymer(s) of the particles present in particular in the dispersion comprises from 10% to 50% by weight of polymerized isobornyl (meth)acrylate and from 50% to 90% by weight of polymerized C1-C4 alkyl (meth)acrylate, relative to the total weight of the combination of the stabilizer(s)+polymer(s) of the particles.

Preferentially, the combination of the stabilizer(s)+polymer(s) of the particles present in particular in the dispersion comprises from 15% to 30% by weight of polymerized isobornyl (meth)acrylate and from 70% to 85% by weight of polymerized C1-C4 alkyl (meth)acrylate, relative to the total weight of the combination of the stabilizer(s)+polymer(s) of the particles.

Preferably, the stabilizer(s) are soluble in the hydrocarbon-based oil, in particular soluble in isododecane.

According to a theory which should not limit the scope of the present invention, the inventors put forward the hypothesis that the surface stabilization of the C₁-C₄ alkyl (meth)acrylate polymer particles results from a phenomenon of surface adsorption of the stabilizer onto the C₁-C₄ alkyl (meth)acrylate polymer particles.

When the polymer particles are provided in the composition in the form of a pre-prepared dispersion, the oily medium of this polymer dispersion comprises a first hydrocarbon-based oil. Reference may be made to that which has been indicated previously concerning this oil as regards its nature.

Advantageously, the hydrocarbon-based oil is apolar and preferably chosen from hydrocarbon-based oils containing from 8 to 16 carbon atoms, in particular the apolar oils described previously.

Preferentially, the hydrocarbon-based oil is isododecane.

The polymer particles, in particular in the dispersion, preferably have an average size, especially a number-average size, ranging from 50 to 500 nm, especially ranging from 75 to 400 nm and better still ranging from 100 to 250 nm.

In general, a dispersion of polymer particles that is suitable for use in the invention may be prepared in the following manner, which is given as an example.

The polymerization may be performed in dispersion, i.e. by precipitation of the polymer during formation, with protection of the formed particles with a stabilizer.

In a first step, the stabilizing polymer is prepared by mixing the constituent monomer(s) of the stabilizing polymer, with a radical initiator, in a solvent known as the synthesis solvent, and by polymerizing these monomers. In a second step, the constituent monomer(s) of the polymer of the particles are added to the stabilizing polymer formed and polymerization of these added monomers is performed in the presence of the radical initiator.

When the non-aqueous medium is a non-volatile hydrocarbon-based oil, the polymerization may be performed in an apolar organic solvent (synthesis solvent), followed by adding the non-volatile hydrocarbon-based oil (which should be miscible with said synthesis solvent) and selectively distilling off the synthesis solvent.

A synthesis solvent which is such that the monomers of the stabilizing polymer and the free-radical initiator are soluble therein, and the polymer particles obtained are insoluble therein, so that they precipitate therein during their foiiiiation, is thus chosen.

In particular, the synthesis solvent may be chosen from alkanes such as heptane or cyclohexane.

When the non-aqueous medium is a volatile hydrocarbon-based oil, the polymerization may be performed directly in said oil, which thus also acts as synthesis solvent. The monomers should also be soluble therein, as should the free-radical initiator, and the polymer of the particles obtained should be insoluble therein.

The monomers are preferably present in the synthesis solvent, before polymerization, in a proportion of 5-20% by weight. The total amount of monomers may be present in the solvent before the start of the reaction, or part of the monomers may be added gradually as the polymerization reaction proceeds.

The free-radical initiator may especially be azobisisobutyronitrile or tert-butyl peroxy-2-ethylhexanoate.

The polymerization may be performed at a temperature ranging from 70 to 110° C.

The polymer particles are surface-stabilized, when they are formed during the polymerization, by means of the stabilizer.

The stabilization may be performed by any known means, and in particular by direct addition of the stabilizer, during the polymerization.

The stabilizer is preferably also present in the mixture before polymerization of the monomers of the polymer of the particles. However, it is also possible to add it continuously, especially when the monomers of the polymer of the particles are also added continuously.

From 10% to 30% by weight and preferably from 15% to 25% by weight of stabilizer may be used relative to the total weight of monomers used (stabilizer+polymer of the particles).

The polymer particle dispersion advantageously comprises from 30% to 65% by weight and preferably from 40% to 60% by weight of solids, relative to the total weight of the dispersion.

Moreover, the composition according to the invention advantageously comprises a content of stabilized polymer particles, described previously, of between 5% and 40% by weight, more particularly from 8% to 30% by weight and preferably from 10% to 25% by weight, relative to the total weight of the composition (content expressed as solids).

Moreover, the composition according to the invention advantageously comprises a content of dispersion, described previously, of between 10% and 60% by weight and more particularly from 15% to 45% by weight, relative to the total weight of the composition.

### Dyestuffs

The compositions in accordance with the invention may comprise at least one dyestuff.

This (or these) dyestuff(s) are preferably chosen from pulverulent substances, liposoluble dyes and water-soluble dyes, and mixtures thereof.

Preferably, the compositions according to the invention comprise at least one pulverulent dyestuff. The pulverulent dyestuffs may be chosen from pigments and nacres, and preferably from pigments.

The pigments may be white or coloured, mineral and/or organic, and coated or uncoated. Among the mineral pigments, mention may be made of metal oxides, in particular titanium dioxide, optionally surface-treated, zirconium, zinc or cerium oxide, and also iron, titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the pigments contained in the compositions according to the invention are chosen from metal oxides. More preferentially, the pigments contained in the compositions according to the invention are chosen from iron oxides, such as especially those sold under the name Sunpuro Black Iron Oxide C33-7001® by the company Sun.

Thus, according to a particular embodiment, a composition according to the invention also comprises at least one dyestuff, the dyestuff(s) preferably being chosen from pulverulent materials, in particular pigments, more particularly from metal oxides such as iron oxides.

These dyestuffs may be present in a content ranging from 0.01% to 30% by weight relative to the total weight of the composition and in particular from 1% to 22% by weight relative to the total weight of the composition.

Preferably, the dyestuff(s) are chosen from one or more metal oxides that are present in a content of greater than or equal to 1% by weight relative to the total weight of the composition, and advantageously inclusively between 3% and 22% by weight relative to the total weight of the composition.

### Additives

The compositions according to the invention may also comprise any cosmetic active agent, such as active agents chosen from an additional volatile or non-volatile silicone oil, fillers, fibres, antioxidants, preserving agents, fragrances, bactericidal active agents, neutralizers, emollients, moisturizers, trace elements, softeners, sequestrants, acidifying or basifying agents, hydrophilic or lipophilic active agents, coalescers and vitamins, and mixtures thereof.

It is a matter of routine operations for a person skilled in the art to adjust the nature and the amount of the additives present in the compositions in accordance with the invention such that the desired cosmetic properties thereof are not thereby affected.

According to a preferred embodiment, a composition of the invention is in the form of a product for the eyelashes, in particular a mascara.

According to another embodiment, a composition of the invention may advantageously be in the form of a product for the eyebrows.

Preferably, a composition according to the invention is in the foils of a composition for caring for and/or making up keratin fibres, in particular the eyelashes, preferably in the form of a mascara.

Such compositions are especially prepared according to the general knowledge of a person skilled in the art.

Throughout the description, including the claims, the term “comprising a” should be understood as being synonymous with “comprising at least one”, unless otherwise specified.

The teams “between . . . and . . . ” and “ranging from . . . to . . . ” should be understood as being inclusive of the limits, unless otherwise specified.

In the description and the examples, the percentages are percentages by weight, unless otherwise indicated. The percentages are thus given on a weight basis relative to the total weight of the composition. The ingredients are mixed in the order and under the conditions that are easily deteunined by those skilled in the art.

The invention is illustrated in greater detail by the examples presented below.

### I. EXAMPLES OF PREPARATION OF DISPERSIONS

### Example 1

In a first step, 1300 g of isododecane, 337 g of isobornyl acrylate, 28 g of methyl acrylate and 3.64 g of tert-butyl peroxy-2-ethylhexanoate (Trigonox 21S from Akzo) were placed in a reactor. The isobornyl acrylate/methyl acrylate mass ratio is 92/8. The mixture was heated at 90° C. under argon with stirring.

After 2 hours of reaction, 1430 g of isododecane were added to the reactor feedstock and the mixture was heated to 90° C.

In a second step, a mixture of 1376 g of methyl acrylate, 1376 g of isododecane and 13.75 g of Trigonox 21S were run in over 2 hours 30 minutes, and the mixture was left to react for 7 hours. 3.3 litres of isododecane were then added and part of the isododecane was evaporated off to obtain a solids content of 50% by weight.

A dispersion of methyl acrylate particles stabilized with a statistical copolymer stabilizer containing 92% isobornyl acrylate and 8% methyl acrylate in isododecane was obtained.

The oily dispersion contains in total (stabilizer+particles) 80% methyl acrylate and 20% isobornyl acrylate.

The polymer particles of the dispersion have a number-average size of about 160 nm.

The dispersion is stable after storage for 7 days at room temperature (25° C.).

### Example 2

A dispersion of polymer in isododecane was prepared according to the preparation method of Example 1, using:

Step 1: 275.5 g of isobornyl acrylate, 11.6 g of methyl acrylate, 11.6 g of ethyl acrylate, 2.99 g of Trigonox 21, 750 g of isododecane; followed by addition, after reaction, of 750 g of isododecane.

Step 2: 539.5 g of methyl acrylate, 539.5 g of ethyl acrylate, 10.8 g of Trigonox 21S, 1079 g of isododecane. After reaction, addition of 2 litres of isododecane and evaporation to obtain a solids content of 35% by weight.

A dispersion in isododecane of methyl acrylate/ethyl acrylate (50/50) copolymer particles stabilized with an isobornyl acrylate/methyl acrylate/ethyl acrylate (92/4/4) statistical copolymer stabilizer was obtained.

The oily dispersion contains in total (stabilizer+particles) 40% methyl acrylate, 40% ethyl acrylate and 20% isobornyl acrylate.

The dispersion is stable after storage for 7 days at room temperature (25° C.).

### Example 3

A dispersion of polymer in isododecane was prepared according to the preparation method of Example 1, using:

Step 1: 315.2 g of isobornyl acrylate, 12.5 g of methyl acrylate, 12.5 g of ethyl acrylate, 3.4 g of Trigonox 21, 540 g of isododecane, 360 g of ethyl acetate; followed by addition, after reaction, of 540 g of isododecane and 360 g of ethyl acetate.

Step 2: 303 g of methyl acrylate, 776 g of ethyl acrylate, 157 g of acrylic acid, 11 g of Trigonox 21S, 741.6 g of isododecane and 494.4 g of ethyl acetate. After reaction, addition of 3 litres of an isododecane/ethyl acetate mixture (60/40 weight/weight) and total evaporation of the ethyl acetate and partial evaporation of the isododecane to obtain a solids content of 44% by weight.

A dispersion in isododecane of methyl acrylate/ethyl acrylate/acrylic acid (24.5/62.8/12.7) copolymer particles stabilized with an isobornyl acrylate/methyl acrylate/ethyl acrylate (92/4/4) statistical copolymer stabilizer was obtained.

The oily dispersion contains in total (stabilizer +particles) 10% acrylic acid, 20% methyl acrylate, 50% ethyl acrylate and 20% isobornyl acrylate.

The dispersion is stable after storage for 7 days at room temperature (25′C).

### Example 4

A dispersion of polymer in isododecane was prepared according to the preparation method of Example 1, using:

Step 1: 315.2 g of isobornyl acrylate, 12.5 g of methyl acrylate, 12.5 g of ethyl acrylate, 3.4 g of Trigonox 21, 540 g of isododecane, 360 g of ethyl acetate; followed by addition, after reaction, of 540 g of isododecane and 360 g of ethyl acetate.

Step 2: 145 g of methyl acrylate, 934 g of ethyl acrylate, 157 g of acrylic acid, 12.36 g of Trigonox 21S, 741.6 g of isododecane and 494.4 g of ethyl acetate. After reaction, addition of 3 litres of an isododecane/ethyl acetate mixture (60/40 weight/weight) and total evaporation of the ethyl acetate and partial evaporation of the isododecane to obtain a solids content of 44% by weight.

A dispersion in isododecane of methyl acrylate/ethyl acrylate/acrylic acid (11.7/75.6/12.7) copolymer particles stabilized with an isobornyl acrylate/methyl acrylate/ethyl acrylate (92/4/4) statistical copolymer stabilizer was obtained.

The oily dispersion contains in total (stabilizer +particles) 10% acrylic acid, 10% methyl acrylate, 60% ethyl acrylate and 20% isobornyl acrylate.

The dispersion is stable after storage for 7 days at room temperature (25° C.).

### Example 5

A dispersion of polymer in isododecane was prepared according to the preparation method of Example 1, using:

Step 1: 48 g of isobornyl acrylate, 2 g of methyl acrylate, 2 g of ethyl acrylate, 0.52 g of Trigonox 21, 57.6 g of isododecane, 38.4 g of ethyl acetate; followed by addition, after reaction, of 540 g of isododecane and 360 g of ethyl acetate.

Step 2: 98 g of methyl acrylate, 73 g of ethyl acrylate, 25 g of maleic anhydride, 1.96 g of Trigonox 21S, 50.4 g of isododecane and 33.60 g of ethyl acetate. After reaction, addition of 1 litre of an isododecane/ethyl acetate mixture (60/40 weight/weight) and total evaporation of the ethyl acetate and partial evaporation of the isododecane to obtain a solids content of 46.2% by weight.

A dispersion in isododecane of methyl acrylate/ethyl acrylate/maleic anhydride (50/37.2/12.8) copolymer particles stabilized with an isobornyl acrylate/methyl acrylate/ethyl acrylate (92/4/4) statistical copolymer stabilizer was obtained.

The oily dispersion contains in total (stabilizer+particles) 10% maleic anhydride, 30% methyl acrylate, 40% ethyl acrylate and 20% isobornyl acrylate.

The dispersion is stable after storage for 7 days at room temperature (25° C.).

### Example 6

A dispersion of polymer in isododecane was prepared according to the preparation method of Example 1, using:

Step 1: 48.5 g of isobornyl methacrylate, 4 g of methyl acrylate, 0.52 g Trigonox 21, 115 g of isododecane; followed by addition, after reaction, of 80 g of isododecane.

Step 2: 190 g of methyl acrylate, 1.9 g of Trigonox 21S, 190 g of isododecane. After reaction, addition of 1 litre of isododecane and partial evaporation of the isododecane to obtain a solids content of 48% by weight.

A dispersion in isododecane of methyl acrylate polymer particles stabilized with an isobornyl methacrylate/methyl acrylate (92/8) statistical copolymer stabilizer was obtained.

The oily dispersion contains in total (stabilizer+particles) 80% methyl acrylate and 20% isobornyl methacrylate.

The dispersion is stable after storage for 7 days at room temperature (25° C.).

### II. COMPOSITION EXAMPLES: MASCARAS

Mascara formulations in accordance with the invention (compositions 1 and 2) or not in accordance with the invention (compositions 3 to 8) are prepared as described below.

To prepare phase B, the hydrophilic gelling agent is added to water in a heating pan with stirring at 70° C. until a homogeneous mixture is obtained. The stirring is adjusted so as not to incorporate air into the mixture.

The rest of the ingredients of phase B are then added at room temperature.

The components of phase A are weighed out in a heating pan and stirred with a Rayneri blender, at 90-95° C.

Once the gels have been prepared and are homogeneous, the two phases are mixed together with a Rayneri blender at room temperature (25° C.).

For the compositions according to the invention, a homogeneous black composition forms.

The composition is prepared using the weight proportions described below. The percentages are given on a weight basis relative to the total weight of the composition.

| | | Composition 1 | Composition 2 |
|---|---|---|---|
| | | according to the | according to the |
| Phase | Compounds | invention | invention |
| Phase A | (Methyl acrylate)-co-(isobornyl acrylate) | 35.00% | 31.22% |
| | (80.7/19.3) copolymer dissolved in | | |
| | isododecane according to Preparation Example | | |
| | 1 described previously | | |
| | Hydrogenated styrene/isoprene copolymer | 4.90% | 5.18% |
| | (Kraton ® G1701 EU sold by the company | | |
| | Kraton Polymers) | | |
| | Hydrogenated styrene/methylstyrene/indene | 8.40% | 10.43% |
| | copolymer (Regalite ® R1100 CG Hydrocarbon | | |
| | Resin sold by the company Eastman Chemical) | | |
| | Glyceryl isostearate (Peceol Isostéarique sold | — | — |
| | by the company Gattefosse) | | |
| | Iron oxides/CI77499 (Sunpuro Black Iron | 4.90% | 8.40% |
| | Oxide C33-7001 sold by the company Sun) | | (preground in |
| | | | isododecane) |
| | Phenoxyethanol (Sepicide LD sold by the | 0.35% | 0.35% |
| | company SEPPIC) | | |
| | Isododecane sold by the company Ineos | qs 100 | qs 100 |
| Phase B | Steareth-100/PEG 136/HDI copolymer | 3.00% | 3.00% |
| | (hexamethyl diisocyanate) (Rheolate ® FX 1100 | | |
| | sold by the company Elementis) | | |
| | Pentylene glycol (616751 Hydrolite ®-5 sold by | 3.00% | 3.00% |
| | the company Symrise) | | |
| | Denatured alcohol (Ethanol SDA 40B 200 | 0.90% | 0.90% |
| | proof sold by the company Sasol) | | |
| | Phenoxyethanol (Neolone PH 100 Preservative | 0.50% | 0.50% |
| | sold by the company Dow Chemical) | | |
| | Microbiologically clean deionized water | 22.60% | 22.60% |

| | | Composition 3 | Composition 4 | Composition 5 |
|---|---|---|---|---|
| | | Outside the | Outside the | Outside the |
| Phase | Compounds | invention | invention | invention |
| Phase A | (Methyl acrylate)-co- | 35.00% | 35.00% | 35.00% |
| | (isobornyl acrylate) | | | |
| | (80.7/19.3) copolymer | | | |
| | dissolved in isododecane | | | |
| | according to Preparation | | | |
| | Example 1 described | | | |
| | previously | | | |
| | Hydrogenated | 4.90% | 4.90% | 4.90% |
| | styrene/isoprene copolymer | | | |
| | (Kraton ® G1701 EU sold by | | | |
| | the company Kraton | | | |
| | Polymers) | | | |
| | Hydrogenated | — | 10.00% | 8.40% |
| | styrene/methylstyrene/indene | | | |
| | copolymer (Regalite ® R1100 | | | |
| | CG Hydrocarbon Resin sold | | | |
| | by the company Eastman | | | |
| | Chemical) | | | |
| | Acrylic acid/isobutyl | — | — | — |
| | acrylate/isobornyl acrylate | | | |
| | copolymer (Mexomer PAS | | | |
| | sold by the company | | | |
| | Chimex) | | | |
| | Iron oxides/CI77499 | 4.90% | 4.90% | 4.90% |
| | (Sunpuro Black Iron Oxide | | | |
| | C33-7001 sold by the | | | |
| | company Sun) | | | |
| | Phenoxyethanol (Sepicide | 0.35% | 0.35% | 0.35% |
| | LD sold by the company | | | |
| | SEPPIC) | | | |
| | Isododecane sold by the | qs 100 | qs 100 | qs 100 |
| | company Ineos | | | |
| Phase B | Steareth-100/PEG 136/HDI | 3.00% | 3.00% | — |
| | copolymer (hexamethyl | | | |
| | diisocyanate) (Rheolate ® FX | | | |
| | 1100 sold by the company | | | |
| | Elementis) | | | |
| | Hydroxyethyl | — | — | — |
| | acrylate/sodium | | | |
| | acryloyldimethyltaurate | | | |
| | copolymer (Sepinov sold by | | | |
| | the company SEPPIC) | | | |
| | Hydroxyethylcellulose | — | — | 1.20% |
| | (HEC) | | | |
| | Pentylene glycol (616751 | — | — | 0.90% |
| | Hydrolite ®-5 sold by the | | | |
| | company Symrise) | | | |
| | Denatured alcohol (Ethanol | 0.90% | 0.90% | 0.90% |
| | SDA 40B 200 proof sold by | | | |
| | the company Sasol) | | | |
| | Phenoxyethanol (Neolone | 0.50% | 0.50% | 0.15% |
| | PH 100 Preservative sold by | | | |
| | the company Dow Chemical) | | | |
| | Microbiologically clean | 25.60% | 22.60% | 26.85% |
| | deionized water | | | |
| | | Composition 6 | Composition 7 | Composition 8 |
| | | Outside the | Outside the | Outside the |
| Phase | Compounds | invention | invention | invention |
| Phase A | (Methyl acrylate)-co- | 31.10% | — | 21.70% |
| | (isobornyl acrylate) | | | |
| | (80.7/19.3) copolymer | | | |
| | dissolved in isododecane | | | |
| | according to Preparation | | | |
| | Example 1 described | | | |
| | previously | | | |
| | Hydrogenated | 5.18% | 5.18% | 4.90% |
| | styrene/isoprene copolymer | | | |
| | (Kraton ® G1701 EU sold by | | | |
| | the company Kraton | | | |
| | Polymers) | | | |
| | Hydrogenated | 10.36% | 10.36% | — |
| | styrene/methylstyrene/indene | | | |
| | copolymer (Regalite ® R1100 | | | |
| | CG Hydrocarbon Resin sold | | | |
| | by the company Eastman | | | |
| | Chemical) | | | |
| | Acrylic acid/isobutyl | — | — | 21.7% |
| | acrylate/isobornyl acrylate | | | |
| | copolymer (Mexomer PAS | | | |
| | sold by the company | | | |
| | Chimex) | | | |
| | Iron oxides/CI77499 | 4.90% | 4.90% | 4.90% |
| | (Sunpuro Black Iron Oxide | | (preground | |
| | C33-7001 sold by the | | in | |
| | company Sun) | | isododecane) | |
| | Phenoxyethanol (Sepicide | 0.35% | 0.35% | 0.35% |
| | LD sold by the company | | | |
| | SEPPIC) | | | |
| | Isododecane sold by the | qs 100 | qs 100 | qs 100 |
| | company Ineos | | | |
| Phase B | Steareth-100/PEG 136/HDI | — | 3.00% | 3.00% |
| | copolymer (hexamethyl | | | |
| | diisocyanate) (Rheolate ® FX | | | |
| | 1100 sold by the company | | | |
| | Elementis) | | | |
| | Hydroxyethyl | 0.60% | — | — |
| | acrylate/sodium | | | |
| | acryloyldimethyltaurate | | | |
| | copolymer (Sepinov sold by | | | |
| | the company SEPPIC) | | | |
| | Hydroxyethylcellulose | — | — | — |
| | (HEC) | | | |
| | Pentylene glycol (616751 | 0.90% | 3.00% | 3.00% |
| | Hydrolite ®-5 sold by the | | | |
| | company Symrise) | | | |
| | Denatured alcohol (Ethanol | 0.90% | 0.90% | 0.90% |
| | SDA 40B 200 proof sold by | | | |
| | the company Sasol) | | | |
| | Phenoxyethanol (Neolone | 0.15% | 0.50% | 0.50% |
| | PH 100 Preservative sold by | | | |
| | the company Dow Chemical) | | | |
| | Microbiologically clean | 27.00% | 22.60% | 22.60% |
| | deionized water | | | |

The textures of the compositions obtained are evaluated macroscopically and microscopically with a Leica DMLB microscope and a Leica ×10 objective lens.

Compositions 1 and 2 (in accordance with the invention) form a macroscopically homogeneous mixture in which observation by microscope reveals that the oily phase and the aqueous phase are both homogeneous.

Compositions 3 to 8 (comparative) are compositions whose film is matt once dry.

### Measurement of the Gloss

A wet product corresponding to each of the compositions 1 to 8 and given in the above tables is spread onto a matt contrast card using a Byk 150 μm square applicator, and the gloss is then measured on the dry film using a glossmeter at 60°.

The gloss of a deposit resulting from the application of a composition may be commonly measured according to various methods, such as the method using a Byk Micro TRI gloss 20°/60°/85° glossmeter.

Principle of the Measurement Using this Glossmeter

The machine illuminates the sample to be analysed at a certain incidence and measures the intensity of the specular reflection.

The intensity of the reflected light depends on the material and on the angle of illumination. For non-ferrous materials (paint, plastic), the intensity of reflected light increases with the angle of illumination. The rest of the incident light penetrates the material and, depending on the shade of the colour, is either partly absorbed or scattered.

The reflectometer measurement results are not based on the amount of incident light but on a polished black glass standard of defined refractive index.

The measurement is normalized relative to an internal standard and brought to a value out of 100: For this calibration standard, the measurement value is set at 100 gloss units (calibration).

The closer the measured value is to 100, the more glossy the sample. The measurement unit is the Gloss Unit (GU).

The angle of illumination used has a strong influence on the reflectometer value. In order to be able to readily differentiate very glossy and matt surfaces, the standardization has defined three geometries or three measurement domains.

### Test Protocol

a-Spread a coat with a wet thickness of 30 μm of the composition whose mean gloss value it is desired to evaluate onto a Leneta brand contrast card of reference Form 1A Penopac, using an automatic spreader. The coat covers the white background and the black background of the card.

b-Leave to dry for 24 hours at 37° C.

c.-Measure the gloss at 20°, 60° and 85° on the matt white absorbent background (3 measurements) using a Byk Gardner brand glossmeter of reference microTri-Gloss.

The measured values in GU obtained for the various test compositions should then be compared. The lower the value measured, the more matt the deposit.

The results are as follows:

| | Compositions | | | |
|---|---|---|---|---|
| | 1 | 2 | 3 outside the | 4 outside the |
| | invention | invention | invention | invention |
| Measurement | 68.4 | 74.5 | 7.2 | 5 |
| of the gloss | | | | |
| | Compositions | | | |
| | 5 outside the | 6 outside the | 7 outside the | 8 outside the |
| | invention | invention | invention | invention |
| Measurement | 15.32 | 10 | 11.3 | 15.82 |
| of the gloss | | | | |

Compositions 1 and 2 (in accordance with the invention) appear very black and very glossy when compared with compositions 3 to 8 (not in accordance with the invention) which are very sparingly glossy, or even matt.

These comparative tests demonstrate that the presence in an architecture of gel-gel type of at least one oily dispersion that is suitable for use in the invention, of pentylene glycol and of a resin that is suitable for use in the invention is a determining factor in order to be able to obtain the desired effect, namely gloss properties and persistence of the gloss after the application of a composition according to the invention to keratin materials and more particularly to keratin fibres.

In addition, compositions 1 and 2 according to the invention afford good properties in terms of transfer resistance, comfort and persistence over time.

1. A composition for coating keratin materials, comprising:
at least one aqueous phase gelled with at least one hydrophilic gelling agent, said hydrophilic gelling agent being at least one nonionic associative of polymer; and
at least one oily phase gelled with at least one lipophilic gelling agent, said lipophilic gelling agent being at least one hydrocarbon-based block copolymer; said oily phase also comprising at least one hydrocarbon-based oil and particles of at least one polymer that is surface-stabilized with a stabilizer, the polymer of the particles being a C1-C4 alkyl (meth)acrylate polymer; the stabilizer being an isobomyl (meth)acrylate polymer chosen from isobomyl (meth)acrylate homopolymer and statistical copolymers of isobomyl (meth)acrylate and of C1-C4 alkyl (meth)acrylate present in an isobomyl (meth)acrylate/C1-C4 alkyl (meth)acrylate weight ratio of greater than 4;
said phases form therein a macroscopically homogeneous mixture; and
said composition also comprising at least one hydrocarbon-based resin with a number-average molecular weight of less than or equal to 10 000 g/mol and pentylene glycol.

2. The composition according to claim 1, wherein said particles are in dispersion in said non-aqueous medium containing at least one hydrocarbon-based oil.

3. Tho composition according to claim 1, wherein the hydrocarbon-based oil(s) are present in the composition at a concentration ranging from 20% to 60% by weight relative to the total weight of the composition, the hydrocarbon-based oil(s) being apolar.

4. The composition according to claim 1, wherein the polymer particle(s) are present in an amount ranging from 5% to 40% by weight, relative to the total weight of the composition.

5. The composition according to claim 1, wherein the polymer(s) of the particles is a methyl acrylate and/or ethyl acrylate polymer(s).

6. The composition according to claim 1, wherein the polymer(s) of the particles comprise an ethylenically unsaturated acid monomer or the anhydride chosen from (meth)acrylic acid, maleic acid, and maleic anhydride thereof.

7. The composition according to claim 1, wherein the polymer(s) of the particles comprise from 80% to 100% by weight of C₁-C₄ alkyl (meth)acrylate and from 0% to 20% by weight of ethylenically unsaturated acid monomer, relative to the total weight of the polymer, the polymer(s) of the particles being chosen from:
methyl acrylate homopolymers
ethyl acrylate homopolymers
methyl acrylate/ethyl acrylate copolymers
methyl acrylate/ethyl acrylate/acrylic acid copolymers
methyl acrylate/ethyl acrylate/maleic anhydride copolymers
methyl acrylate/acrylic acid copolymers
ethyl acrylate/acrylic acid copolymers
methyl acrylate/maleic anhydride copolymers
ethyl acrylate/maleic anhydride copolymers.

8. The compositon according to claim 1, wherein the stabilizer(s) is a statistical copolymer(s) of isobornyl (meth)acrylate and of C1-C4 alkyl (meth)acrylate present in an isobornyl (meth)acrylate/C1-C4 alkyl (meth)acrylate weight ratio of greater than or equal to 5.

9. The composition according to claim 1, wherein the stabilizer(s) are chosen from:
isobornyl acrylate homopolymers
statistical copolymers of isobornyl acrylate/methyl acrylate
statistical copolymers of isobornyl acrylate/methyl acrylate/ethyl acrylate
statistical copolymers of isobornyl methacrylate/methyl acrylate.

10. The composition according to claim 1, characterized wherein the combination of the stabilizer(s)+polymer(s) of the particles present in the dispersion comprises from 10% to 50% by weight of polymerized isobornyl (meth)acrylate and from 50% to 90% by weight of polymerized C1-C4 alkyl (meth)acrylate, relative to the total weight of the combination of the stabilizer+polymer of the particles.

11. The composition according to claim 1, comprising from 3% to 30%; by weight of hydrocarbon-based resin(s), relative to the total weight of the composition.

12. The composition according to claim 1, wherein said hydrocarbon-based resin(s) are present totally or partially, and -solely, in the gelled oily phase.

13. The composition according to claim 1, wherein said hydrocarbon-based resin(s) are chosen from indene hydrocarbon-based resins, aliphatic pentadiene resins and mixtures thereof.

14. The composition according to claim 1, wherein said hydrocarbon-based resin(s) are chosen from hydrogenated indene/methylstyrene/styrene copolymers.

15. The composition according to claim 1, comprising, as lipophilic gelling agent, at least one copolymer bearing styrene blocks and bearing ethylene/C₃-C₄ alkylene blocks.

16. The composition according to claim 1, comprising, as hydrophilic gelling agent, at least one fatty-chain nonionic polyurethane polyether.

17. The composition according to claim 1, comprising a water content at least equal to 10% by weight weight, relative to the total weight of the composition.

18. The composition according to claim 1, further comprising at least one dyestuff.

19. The compositon according to claim 1, wherein the composition is in a form for caring for and/or making up keratin fibers.

20. The compisition according to claim 1, wherein the composition is free of surfactants.

21. A process for preparing a composition, for coating keratin materials, comprising at least one step of mixing:
an aqueous phase gelled with at least one hydrophilic gelling agent, said hydrophilic gelling agent being at least one nonionic associative polymer; and
at least one oily phase gelled with at least one lipophilic gelling agent, said lipophilic gelling agent being at least one hydrocarbon-based block copolymer; said oily phase also comprising at least one hydrocarbon-based oil and particles of at least one polymer that is surface-stabilized with a stabilizer, the polymer of the particles being a C1-C4 alkyl (meth)acrylate polymer; the stabilizer being an isobornyl (meth)acrylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4;
under conditions suitable for obtaining a macroscopically homogeneous mixture;
said composition also comprising at least one hydrocarbon-based resin with a number-average molecular weight of less than or equal to 10 000 g/mol and pentylene glycol.

22. A cosmetic method for making up and/or caring for a keratin material, comprising at least one step which consists in applying to said keratin materials a composition comprising:
at least one aqueous phase gelled with at least one hydrophilic gelling agent said hydrophylic gellling agent being at least one nonionic associative polymer; and
at least one oily phase gelled with at least one lipophilic gelling agent, said lipophilic gelling agent being at least one hvdrocarbon-based block copolymer; said oily phase also comprising at least one hydrocarbon-based oil and particles of at least one polymer that is surface-stabilized with a stabilizer, the polymer of the particles being C1-C4 alkyl (meth)acrylate polymer; the stabilizer being an isobomyl (meth)actylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl(meth)acrylate weight ratio of greater than 4:
said phases forming therein a macroscopically homogeneous mixture; and
said composition also comprising at least one hydrocarbon-based resin with a number-average molecular weight of less than or equal to 10 000 g/niol and pentylene glycol,

23. A cosmetic method, for making up and/or caring for keratin materials, comprising at least the application to said keratin materials of a macroscopically homogeneous composition obtained by extemporaneous mixing, before application or at the time of application to said keratin materials, of at least one aqueous phase gelled with at least one hydrophilic gelling agent, said hydrophilic gelling agent being at least one nonionic associative polymer, and at least one oily phase gelled with at least one lipophilic gelling agent, said lipophilic gelling agent being at least one hydrocarbon-based block copolymer, said oily phase also comprising at least one hydrocarbon-based oil and particles of at least one polymer surface-stabilized with a stabilizer, the polymer of the particles being a C1-C4 alkyl (meth)acrylate polymer; the stabilizer being an isobornyl (meth)acrylate polymer chosen from isobornyl (meth)acrylate homopolymer and statistical copolymers of isobornyl (meth)acrylate and of a C1-C4 alkyl (meth)acrylate present in an isobornyl (meth)acrylate/C1-C4 alkyl (meth)acrylate weight ratio of greater than 4; said composition also comprising at least one hydrocarbon-based resin with a number-average molecular weight of less than or equal to 10 000 g/mol; and pentylene glycol.

24. A process for preparing a mascara composition, wherein at least one stage of the process is carried out using a dispersion of particles of at least one polymer that is surface-stabilized with a stabilizer in a non-aqueous medium containing at least one hydrocarbon-based oil, the polymer of the particles being a C1-C4 alkyl (meth)acrylate polymer; the stabilizer being an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/C1-C4 alkyl (meth)acrylate weight ratio of greater than 4.
